(19) United States
(12) Patent Application Publication
(10) Pub. No.: US 2014/0371649 A1
(43) Pub. Date: Dec. 18, 2014

(54) ORTHOTIC DEVICE FOR RELIEVING TOE PAIN

(71) Applicant: JODI L. TALLEY, Sioux Falls, SD (US)

(72) Inventor: JODI L. TALLEY, Sioux Falls, SD (US)

(21) Appl. No.: 13/916,253

(22) Filed: Jun. 12, 2013

(51) Int. Cl. A61F 5/01

(52) U.S. Cl. CPC A61F 5/019
USPC 602/30

(57) ABSTRACT

A foot pain relief device may comprise a pad configured be positioned under at least a portion of the first metatarsophalangeal joint and a portion of the phalange of the hallux. The pad has an upper surface for positioning toward the foot and may include a first portion configured for positioning under the first metatarsophalangeal joint of the foot. The first portion has central and peripheral regions, with a section of the upper surface on the central portion being depressed and a section of the upper surface on the peripheral portion being raised. The pad includes a second portion configured for positioning under the phalange of the hallux when the first portion is positioned under the first metatarsophalangeal joint. A region of the upper surface formed by the second portion is relatively higher than a region of the upper surface of the pad formed by the first portion.

### BACKGROUND

### Field

The present disclosure relates to orthotic devices and more particularly pertains to a new orthotic device for relieving toe pain.

### SUMMARY

In one aspect, the disclosure relates to a foot pain relief device which may comprise a pad configured be positioned under at least a portion of the first metatarsophalangeal joint and a portion of the phalange of the hallux of the foot. The pad may have an upper surface for positioning toward the foot and a lower surface for positioning away from the foot. The pad may include a first portion configured for positioning under the first metatarsophalangeal joint of the foot, and the first portion may have a central region and a peripheral region about at least a portion of the section of the central region. A section of the upper surface on the central portion may be relatively depressed and a section of the upper surface on the peripheral portion may be relatively raised. A second portion may be configured for positioning under a portion of the phalange of the hallux when the first portion is positioned under the first metatarsophalangeal joint of the foot. A region of the upper surface of the pad formed by the second portion may be relatively higher than a region of the upper surface of the pad formed by the first portion.

In another aspect, the disclosure relates to a foot pain relief device which may comprise a pad configured be positioned under at least a portion of the first metatarsophalangeal joint and a portion of the phalange of the hallux of the foot. The pad may have an upper surface for positioning toward the foot and a lower surface for positioning away from the foot. The pad may include a first portion configured for positioning under the first metatarsophalangeal joint of the foot, and the first portion may have a central region and a peripheral region about at least a portion of the section of the central region. A section of the upper surface on the central portion may be relatively depressed and a section of the upper surface on the peripheral portion may be relatively raised. The central region of the first portion may have a relatively thinner thickness and the peripheral region of the first portion may have a relatively thicker thickness. A second portion may be configured for positioning under a portion of the phalange of the hallux when the first portion is positioned under the first metatarsophalangeal joint of the foot. A region of the upper surface of the pad formed by the second portion may be relatively higher than a region of the upper surface of the pad formed by the first portion. The device may also include a cover positioned over the pad, and may include an upper layer and a lower layer. The lower layer may be secured to the upper layer at a perimeter edge of the cover with the pad being positioned therebetween.

There has thus been outlined, rather broadly, some of the more important elements of the disclosure in order that the detailed description thereof that follows may be better understood, and in order that the present contribution to the art may be better appreciated. There are additional elements of the disclosure that will be described hereinafter and which will form the subject matter of the claims appended hereto.

In this respect, before explaining at least one embodiment or implementation in greater detail, it is to be understood that the scope of the disclosure is not limited in its application to the details of construction and to the arrangements of the components set forth in the following description or illustrated in the drawings. The disclosure is capable of other embodiments and implementations and is thus capable of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present disclosure. It is important, therefore, that the claims be regarded as including such equivalent constructions insofar as they do not depart from the spirit and scope of the present disclosure.

The advantages of the various embodiments of the present disclosure, along with the various features of novelty that characterize the disclosure, are disclosed in the following descriptive matter and accompanying drawings.

### BRIEF DESCRIPTION OF THE DRAWINGS

The disclosure will be better understood and when consideration is given to the drawings and the detailed description which follows. Such description makes reference to the annexed drawings wherein:

FIG. 1 is a schematic top view of a new orthotic device for relieving toe pain according to the present disclosure.

FIG. 2 is a schematic sectional view of the device taken along line 2-2 of FIG. 1.

FIG. 3 is a schematic side sectional view of the device with a foot positioned thereon.

FIG. 4 is a schematic top view of the pad inside a cover, according to an illustrative embodiment.

FIG. 5 is a schematic sectional view taken along line 5-5 of FIG. 4.

FIG. 6 is a schematic top view of the pad mounted on the strap of a securing device, according to an illustrative embodiment.

### DETAILED DESCRIPTION

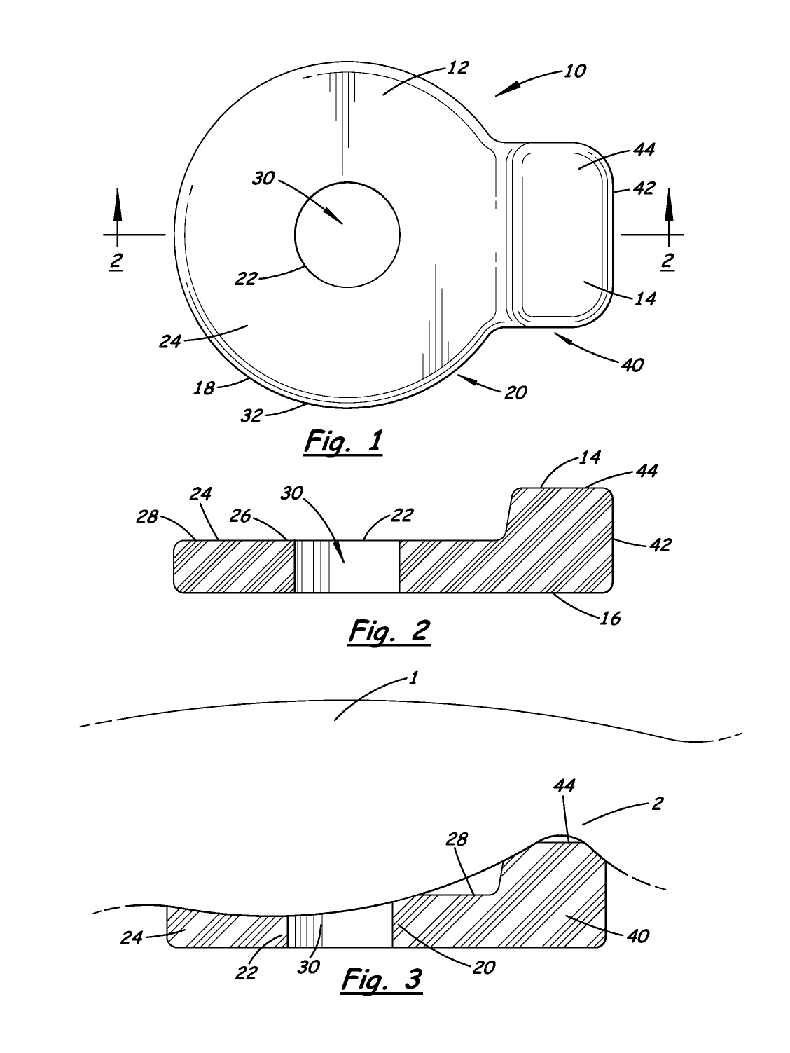

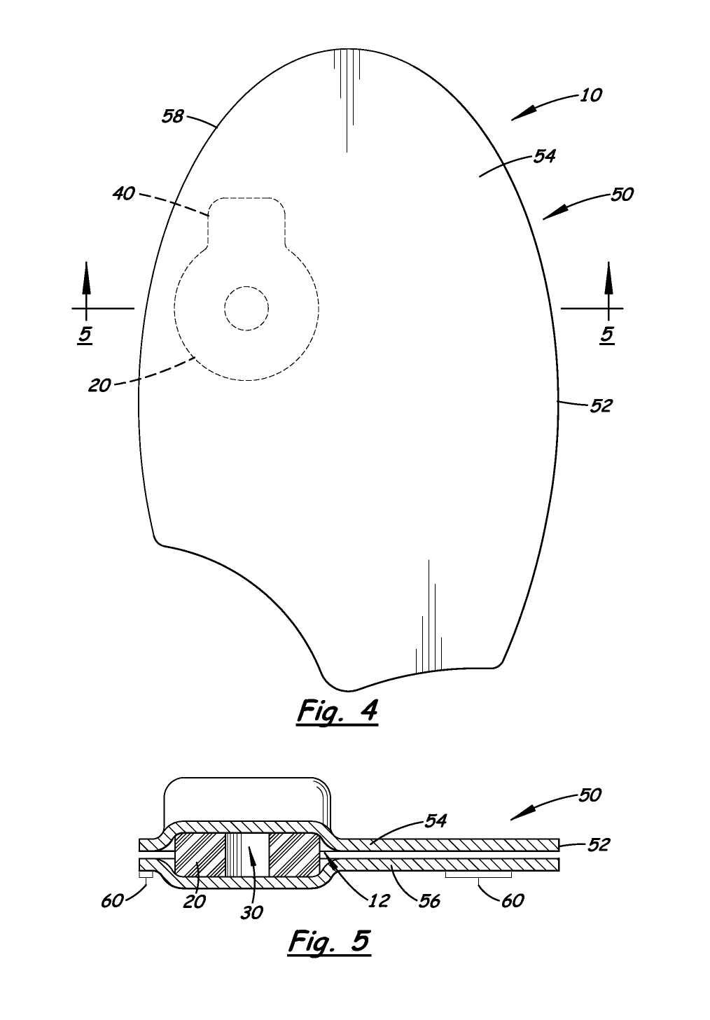

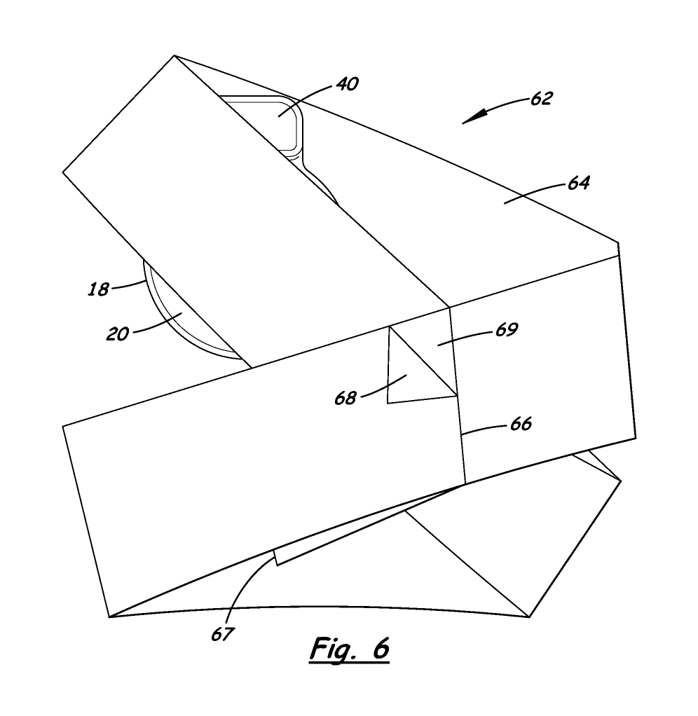

With reference now to the drawings, and in particular to FIGS. 1 through 6 thereof, a new orthotic device for relieving toe pain that embodies the principles and concepts of the disclosed subject matter will be described.

The applicant has recognized that the condition of hallux limitus or hallux rigidus can affect the foot of a person, and cause discomfort when standing for an extended period of time, or walking, to the person and limit the ability of the person to walk or run for long distances without significant pain. The condition affects, at least in part, the joint between the first metatarsophalangeal joint of the foot between the first metatarsal and the phalange of the hallux, or large toe. The applicant has also recognized that an upward lifting of the phalange, even to a small degree, places a small downward bend in the first metatarsophalangeal joint and provides relief to the arthritis sufferer. The applicant has further realized that lifting or raising the first metatarsophalangeal joint (in concert with lifting the first metatarsal) can minimize or erase the pain easing effect, and also cause the upper surface of the foot to be uncomfortably pressed against the inner surface of the upper portion of the footwear. Thus, the applicant has determined that lifting at least the base of the first metatarsal in combination with minimal (if any) lifting of the first metatarsophalangeal joint provides the most relief from arthritis pain in the joint without causing additional discomfort.

The disclosure is thus directed to a foot pain relief device 10 for positioning under a portion of the foot of the user, and typically under at least a portion of the first metatarsophalangeal joint of the foot and at least a portion of the phalange of the hallux, or large toe. The device 10 may be used with an article of footwear, such as a shoe or sandal or even a sock, and may be inserted into an interior of footwear of the user. The device 10 is typically positioned between the foot of the user and the upper surface of the sole or sock liner of the shoe.

The pain relief device 10 may include a pad 12 that is generally configured to rest a portion of the user's foot thereon for therapeutic effect. The pad 12 may be configured for positioning under at least a portion of the first metatarsophalangeal joint 1 and a portion of the phalange of the big toe. The pad 12 may be formed of a resiliently compressible material, such as a foamed material including a closed cell foam, although this material is not critical to the function of the device 10. The pad 12 has an upper surface 14 for positioning toward the foot and a lower surface 16 for positioning away from the foot and toward a sole of the footwear. The pad 12 may also have a peripheral edge 18 that extends about the pad.

In some greater degree of detail, the pad includes a first or anchor portion 20 that may be configured to be positioned under the first metatarsophalangeal joint of the foot. The anchor portion 20 may have a central region 22 and a peripheral region 24 extending about at least a portion of the central region. A section 26 of the upper surface on the central region may be relatively depressed and a section 28 of the upper surface on the peripheral region may be relatively raised, and the central region 22 may have a relatively thinner thickness and the peripheral region 24 may have a relatively thicker thickness. In some embodiments, a section 32 of the peripheral edge 18 that corresponds to the first portion 20 may be substantially circular in shape, although this shape is not critical.

In the most preferred embodiments, the first portion 20 of the pad 12 is configured to substantially maintain a height of the first metatarsophalangeal joint relative to a remainder of the foot without raising the metatarsophalangeal joint, or to the smallest degree possible. In some embodiments, the central region 22 may include an aperture 30 surrounded by the peripheral region to thereby minimize the degree to which the first metatarsophalangeal joint.

The pad 12 may also include a second or lifting portion 40 that may be configured for positioning under a portion of the phalange of the hallux, or the first bone of the big toe extending from the metatarsal joint. The second portion 40 may be positionable under the base of the phalange of the hallux when the first portion 20 is positioned under the first metatarsophalangeal joint of the foot. The second portion 40 may extend outwardly from the first portion 20. A section 42 of the peripheral edge corresponding to the second portion may have an elongated shape, and may be elongated in a direction that is substantially perpendicular to a radius extending outwardly from a center of the central region. The second portion may be oriented tangential to the first portion.

A section 44 of the upper surface 14 of the pad that is on the second portion 40 may be located at a vertical level relatively higher than the section of the upper surface of the pad that is on the first portion, such as the section 28 of the peripheral region. A part of the second portion 40 may have a thicker thickness than the thickness of the first portion at any location thereon.

The foot pain relief device 10 may also include a cover 50 positioned over the pad 12 and have a perimeter edge 52. The cover 50 may be formed of a flexible material to conform to the upper surface of the pad, and the material forming the cover may be continuous between the extents of the perimeter edge without holes. The cover 50 may extend beyond a periphery of the pad and the peripheral edge 18 thereof. The perimeter of the cover 50 may thus define a larger area than an area defined by the periphery of the pad, with the pad being positioned between the upper and lower layers. The cover may include an upper layer 54 and a lower layer 56. The lower layer may be secured to the upper layer at least at the perimeter edge of the cover, and the layers may be secured together at other locations, such as through the aperture 30 of the first portion.

In some embodiments, the shape of the perimeter edge 52 of the cover may generally mirror or follow the peripheral edge of the pad, but typically be larger than the peripheral edge profile. In other embodiments, the perimeter edge 52 may be adapted to the shape of the footwear on which it is to be employed, such as a shoe. The perimeter edge 52 may have an arcuate segment 58 that is shaped similar to a forward portion of the sole of a shoe to fit into the toebox of a shoe. In yet other embodiments, the perimeter edge of the cover may have a shape that does not fall into either of these general categories.

The device 10 may also include an attachment element 60 that is positioned on the cover 50 or the pad 12 to removably secure the cover or the pad or the combination of the cover and pad on the footwear. In some embodiments, the attachment element 60 may comprise a hook and loop fastener component, such as is sold under the tradename VELCRO, and may mate with another hook and loop fastener component that is mounted on the footwear, such as on the sock liner. The attachment element 60 may be bonded, such as by adhesive, or may be mechanically attached, such as by sewing, to the lower layer of the cover or to the pad. In some embodiments, the attachment element may include two pieces of hook and loop fastener components on the cover pad.

The foot pain relief device 10 may further comprise a securing device 62 that is configured to wrap about a portion of the foot of the user, and may be formed of a strap 64 forming a loop to loop about the fore portion of the foot generally adjacent to the first metatarsophalangeal joint. The pad, or the pad with the cover, may be removably secured to the strap 64. The strap 64 may have opposite ends 66, 67, with a first hook and loop fastener component 68 that is attached to one end 66 of the strap and a second hook and loop fastener component 69 that is attached to a medial portion of the strap 64.

Generally, the length of the second portion may range from approximately 1½ to approximately 2½ times the width of the second portion, and the length may be approximately twice the width of the second portion. The length of the second portion may be from approximately 50 percent to approximately 100 percent of the maximum dimension of the first portion, and the width may be approximately ⅔ of the maximum dimension of the second portion.

In some embodiments, the peripheral region 24 of the first portion of the pad 12 may have a thickness in the range of approximately ⅛ inches to ⅜ inches, and the central region 22 of the first portion of the pad may have a thickness from zero to approximately ⅛ inches. The first portion may have a maximum width, or diameter, of between approximately 1 inches and approximately 2½ inches. The second portion 40 of the pad may have a thickness of between approximately ¼ inches and approximately ½ inches. The second portion may have a length of between approximately 1 inch and approximately 2 inches, and a width of between approximately ½ inches and approximately ⅞ inches.

In an illustrative embodiment, the peripheral region 24 of the first portion of the pad 12 may have a thickness of approximately ¼th inches, and the central region 22 of the first portion of the pad may have a thickness approaching zero, if not actually zero where the aperture is located. The first portion may have a maximum width, or diameter, of approximately 2¼ inches. The second portion 40 of the pad may have a thickness of approximately ⅜ inches thick. The second portion may have a length of approximately 1½ inches, and a width of approximately ¾ inches.

It should be appreciated that in the foregoing description and appended claims, that the terms “substantially” and “approximately,” when used to modify another term, mean “for the most part” or “being largely but not wholly or completely that which is specified” by the modified term.

It should also be appreciated from the foregoing description that, except when mutually exclusive, the features of the various embodiments described herein may be combined with features of other embodiments as desired while remaining within the intended scope of the disclosure.

Further, those skilled in the art will appreciate that the steps shown in the drawing figures may be altered in a variety of ways. For example, the order of the steps may be rearranged, substeps may be performed in parallel, shown steps may be omitted, or other steps may be included, etc.

With respect to the above description then, it is to be realized that the optimum dimensional relationships for the parts of the disclosed embodiments and implementations, to include variations in size, materials, shape, form, function and manner of operation, assembly and use, are deemed readily apparent and obvious to one skilled in the art in light of the foregoing disclosure, and all equivalent relationships to those illustrated in the drawings and described in the specification are intended to be encompassed by the present disclosure.

Therefore, the foregoing is considered as illustrative only of the principles of the disclosure. Further, since numerous modifications and changes will readily occur to those skilled in the art, it is not desired to limit the disclosed subject matter to the exact construction and operation shown and described, and accordingly, all suitable modifications and equivalents may be resorted to that fall within the scope of the claims.

I claim:

1. A foot pain relief device comprising:
a pad configured be positioned under at least a portion of the first metatarsophalangeal joint and a portion of the phalange of the hallux of the foot, the pad having an upper surface for positioning toward the foot and a lower surface for positioning away from the foot, the pad including:
a first portion configured for positioning under the first metatarsophalangeal joint of the foot, the first portion having a central region and a peripheral region about at least a portion of the section of the central region, a section of the upper surface on the central portion being relatively depressed and a section of the upper surface on the peripheral portion being relatively raised;
a second portion configured for positioning under a portion of the phalange of the hallux when the first portion is positioned under the first metatarsophalangeal joint of the foot; and
wherein a region of the upper surface of the pad formed by the second portion is relatively higher than a region of the upper surface of the pad formed by the first portion.

2. The device of claim 1 wherein the pad is formed of a resiliently compressible material.

3. The device of claim 1 wherein the first portion is configured to substantially maintain a height of the first metatarsophalangeal joint relative to a remainder of the foot without raising the metatarsophalangeal joint.

4. The device of claim 3 wherein the central region of the first portion has a relatively thinner thickness and the peripheral region of the first portion has a relatively thicker thickness.

5. The device of claim 3 wherein the central region of the first portion includes an aperture surrounded by the peripheral region of the first portion.

6. The device of claim 1 wherein a part of the second portion has a thicker thickness than a thickness of the first portion.

7. The device of claim 1 wherein the pad has a peripheral edge, a section of the peripheral edge corresponding to the first portion being substantially circular in shape.

8. The device of claim 7 wherein a section of the peripheral edge corresponding to the second portion has an elongated shape.

9. The device of claim 1 additionally comprising a cover positioned over the pad.

10. The device of claim 9 wherein the cover includes:
an upper layer; and
a lower layer, the lower layer being secured to the upper layer at a perimeter edge of the cover with the pad being positioned therebetween.

11. The device of claim 9 additionally comprising an attachment element positioned on the cover to removably secure the cover and pad on the footwear.

12. The device of claim 1 additionally comprising a securing device configured to wrap about a portion of the foot of the user and hold the pad in place on the foot.

13. The device of claim 12 wherein the securing device includes a strap forming a loop to loop about the fore portion of the foot adjacent to the first metatarsophalangeal joint, the pad being removably secured to the strap.

14. A foot pain relief device comprising:
a pad configured be positioned under at least a portion of the first metatarsophalangeal joint and a portion of the phalange of the hallux of the foot, the pad having an upper surface for positioning toward the foot and a lower surface for positioning away from the foot, the pad including:
a first portion configured for positioning under the first metatarsophalangeal joint of the foot, the first portion having a central region and a peripheral region about at least a portion of the section of the central region, a section of the upper surface on the central portion being relatively depressed and a section of the upper surface on the peripheral portion being relatively raised, the central region of the first portion having a relatively thinner thickness and the peripheral region of the first portion having a relatively thicker thickness;
a second portion configured for positioning under a portion of the phalange of the hallux when the first portion is positioned under the first metatarsophalangeal joint of the foot, a region of the upper surface of the pad being formed by the second portion is relatively higher than a region of the upper surface of the pad formed by the first portion; and
a cover positioned over the pad, the cover including an upper layer and a lower layer, the lower layer being secured to the upper layer at a perimeter edge of the cover with the pad being positioned therebetween.

15. The device of claim 14 wherein the pad is formed of a resiliently compressible material.

16. The device of claim 14 wherein the central region of the first portion includes an aperture surrounded by the peripheral region of the first portion.

17. The device of claim 14 wherein a part of the second portion has a thicker thickness than a thickness of the first portion.

18. The device of claim 14 additionally comprising an attachment element positioned on the cover to removably secure the cover and pad on the footwear.
